(19) United States
(12) Patent Application Publication
(10) Pub. No.: US 2007/0191826 A1
(43) Pub. Date: Aug. 16, 2007

(54) MATRIX ROUTER

(76) Inventors: Christopher J. Park, Oregonia, OH (US); Salvatore Privitera, Mason, OH (US)

Correspondence Address: COOK, ALEX, MCFARRON, MANZO, CUMMINGS & MEHLER LTD, SUITE 2850, 200 WEST ADAMS STREET, CHICAGO, IL, 60606, US

(21) Appl. No.: 11/457,531

(22) Filed: Jul. 14, 2006

Related U.S. Application Data
(60) Provisional application No. 60/699,664, filed on Jul. 15, 2005.

(51) Int. Cl. A61B 18/18

(52) U.S. Cl. 606/34; 606/41; 607/2; 600/374

(57) ABSTRACT

A surgical router can be used for connecting multiple disposable devices to a single piece of capital equipment, and connecting multiple pieces of capital equipment to a single disposable device. The surgical router also can be used to simplify the workflow for a surgical procedure by allowing multiple tasks to be performed, such as ablation of tissue and pacing of tissue, without requiring switching of handpiece connections.

### PRIORITY

This application claims priority to and the benefit of U.S. provisional application No. 60/699,664 filed on Jul. 15, 2005.

### BACKGROUND

The present invention relates to surgical instruments, with examples relating to ablation devices and systems for controlling such devices. Surgery generally refers to the diagnosis or treatment of injury, deformity, or disease. Surgical devices generally refer to tools which can be used during surgery. In a variety of surgical procedures, it may be desired to remove or cause the destruction of tissue, such as by ablation. Some examples of such procedures include, without limitation, electrical isolation of cardiac tissue to treat atrial fibrillation, ablation of uterine tissue associated with endometriosis, ablation of esophageal tissue associated with Barrett's esophagus, ablation of cancerous liver tissue, and the like. A device which can be used to ablate during surgery is referred to as an ablation surgical device. The foregoing examples are merely illustrative and not exhaustive. While a variety of techniques and devices have been used to ablate or cause lesions in tissue, no one has previously made or used a device in accordance with the present invention.

### BRIEF DESCRIPTION OF DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 is a schematic view of modules in an exemplary embodiment of a matrix router;

FIG. 1A is a schematic view of modules in an alternate exemplary embodiment of a matrix router;

FIG. 2A illustrates a front view of an exemplary matrix router;

FIG. 2B illustrates a rear view of the matrix router of FIG. 2A;

FIG. 3A illustrates a front view of an alternative matrix router;

FIG. 3B illustrates a rear view of the matrix router of FIG. 3A;

FIG. 4A illustrates a left view of the matrix router of FIG. 2A;

FIG. 4B illustrates a right view of the matrix router of FIG. 2A;

FIG. 5 illustrates a front view of an alternative matrix router.

FIG. 6 illustrates a front view of an alternative matrix router.

FIG. 7 illustrates a front view of an alternative matrix router.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

In some embodiments, a surgical router, also referred to as a matrix router, might be used to facilitate the performance of the Maze procedure through bipolar radio frequency ablation. As is well known to one of ordinary skill in the art, the Maze procedure is a procedure used to treat atrial fibrillation, a form of cardiac arrhythmia characterized by a loss of synchrony between the atria and ventricles of the heart. The Maze procedure treats atrial fibrillation through establishing conduction blocks in the heart which serve to stop the formation and conduction of the electrical patterns which are responsible for atrial fibrillation. When using bipolar radio frequency ablation to create the conduction blocks, a surgeon uses a device, such as an isolator transpolar pen (one type of which is disclosed in a U.S. patent application Ser. No. 11/363,707 entitled “Surgical Ablation and Pacing Device”, filed Feb. 28, 2006, the teaching of which is incorporated by reference, by way of example only), an isolator transpolar clamp (one type of which is disclosed in U.S. Pat. No. 6,517,536, the teaching of which is incorporated by reference, by way of example only), or some other surgical device, to deliver bipolar radio frequency energy to cardiac tissue. As bipolar radio frequency energy is applied to the tissue, the outer layers of the tissue may become non-conductive. As the outer layers of the tissue become non-conductive, the bipolar radio frequency energy may begin to pass through deeper and deeper levels of tissue, until eventually the entire area of tissue selected by the surgeon has been ablated, creating a conduction block. Finally, to ensure that a conduction block has been successfully created, the surgeon might test the electrical activity and response of the cardiac tissue using techniques such as pacing, stimulating and sensing. As is well known to those of skill in the art, in this context, pacing refers to applying electrical impulses to cardiac tissue at a rate higher than the patient's current heart rate (e.g., 10 to 20 beats per minute higher), stimulating refers to pacing which is performed at a relatively high rate and sensing refers to the process of monitoring the electrical activity of the contact tissue surface. As an example of the use of those techniques, a surgeon might pace the tissue on the side of a conduction block which is opposite the heart chamber and observe the heart (for example, through visual observation, through observation of a electrocardiogram (ECG), or through some other means) to ensure that the pacing does not change the rate of the patient's heart beat. As an example of the use of sensing, a surgeon might use a tool to sense the electrical activity of a patient's cardiac tissue to ensure that a fibrillatory signal does not cross over a lesion. As an example of stimulating, a surgeon might stimulate cardiac tissue and then observe the vagal (heart rate) response on an ECG. Of course, one or more of those techniques, or other techniques known to those of skill in the art, might be combined in order to verify that a conduction block has been created. Additionally, it will be appreciated that this disclosure does not individually specify each testing technique that can be used, and will describe the use of a matrix router in terms of particular techniques, such as pacing or sensing. As will be clear to one of ordinary skill in the art, the invention is not limited to the use of the testing techniques specifically set forth in the description, and other techniques, such as stimulating, could be substituted for the elaborated techniques without departing from the scope or spirit of the invention.

Because multiple pieces of equipment might be required for performing the Maze procedure, and those pieces of equipment might require different radio frequency (RF) energy generation algorithms, or might use alternative types of energy entirely, it may be desirable for a piece of equipment, such as any of the matrix routers described herein, to allow the integration of surgical devices and to allow multiple disposable devices to be driven by a single piece of capital equipment without switching connections between devices. Further, one with ordinary skill in the art will recognize that a matrix router may be utilized in contexts other than performance of the Maze procedure, such as ablation of uterine tissue associated with endometriosis, ablation of esophageal tissue associated with Barrett's esophagus, ablation of cancerous liver tissue, and other procedures. Additionally, while the illustrative examples set forth below will generally discuss the performance of surgical procedures using bipolar radio frequency energy, it will be immediately apparent to one of ordinary skill in the art that a matrix router may be used with other types of energy, such as ultrasonic energy, mono-polar radio frequency energy, microwave energy, laser energy, or other types of energy. Further, while the description of the Maze procedure set forth above specifically mentions the use of certain tools such as an isolator transpolar pen and isolator transpolar clamp, one of ordinary skill in the art will immediately recognize that other ablation surgical devices might be used to perform the Maze procedure or other surgical procedures. Therefore, the examples presented herein discussing the use of a matrix router are intended to be illustrative only, and are not intended as limiting on the scope of uses or configurations of the matrix router.

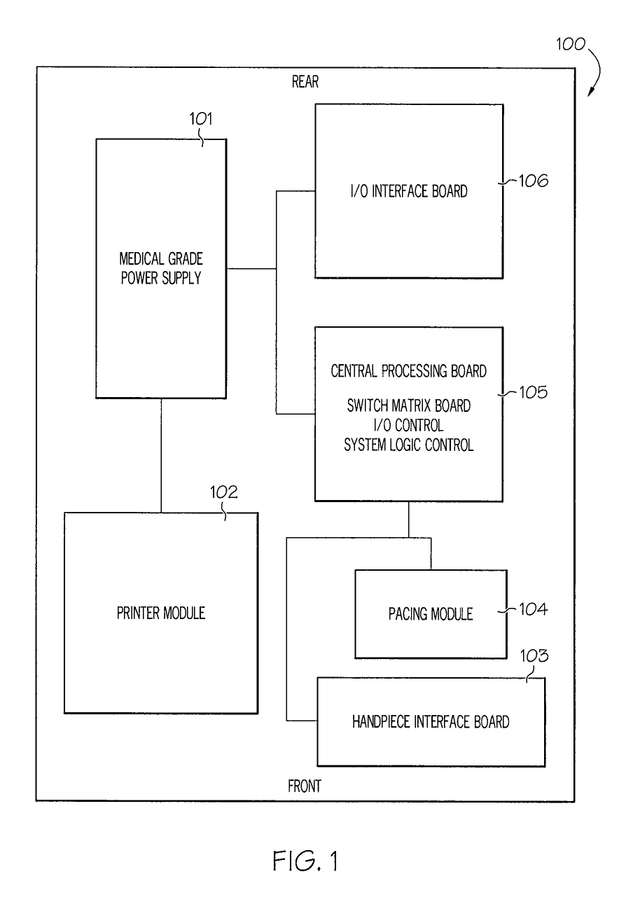

As shown in FIG. 1, the matrix router (100) of the present example comprises an energy generator (101), a printer control module (102), a handpiece interface circuit (103), a pacing module (104), a control circuit (105), and an input/output circuit (106). As used herein, the term “circuit” and variations thereof should be understood to refer any type of electrical equipment, including programmable memory and associated devices. Similarly, the term module should be understood to refer to any portion of a device which performs at least one delimited function, and possibly other functions. It will be immediately apparent to one of ordinary skill in the art that a module might be implemented in circuitry, and that a single circuitry might contain multiple modules. One example of a circuitry which contains multiple modules would be a circuitry comprising memory containing multiple sets of computer instructions wherein each set of computer instructions is dedicated to accomplishing a delimited function. Other module examples will be apparent to those of ordinary skill in the art.

For purposes of illustration, a discussion of how various components and modules depicted in FIG. 1 might operate and/or interact with one another will be set forth. It should be understood that such discussion is intended to be illustrative only of how certain embodiments might function, and is not intended to be limiting on the scope of the invention as a whole. In some embodiments, if a surgeon indicates a desire to use an isolator transpolar pen with the matrix router (100) of this example, the handpiece interface board (103) might send a signal to the central processing board (105) notifying the central processing board (105) that the surgeon wishes to use an isolator transpolar pen in a particular mode, for example, ablation mode. In response to receiving that signal, the central processing board (105) might trigger the energy generator (101) or some external generator (not shown) to supply bi-polar radio frequency energy, which might then be routed to the isolator transpolar pen by the central processing board (105) through the handpiece interface board (103). When the surgeon finishes using the isolator transpolar pen for ablation, he or she might wish to verify the creation of a conduction block, which might be done by pacing. The matrix router (100) could facilitate the process of switching from ablation to pacing through a process comprising the step of sending a signal from the handpiece interface board (103) to the central processing board (105), indicating that the isolator transpolar pen should be used in pacing mode, rather than ablation mode. In response to receiving that signal, the central processing board (105) might activate the pacing module (104), and might additionally cause a connection between the pacing module (104) and the isolator transpolar pen to be established, so that the surgeon could test to verify the establishment of a conduction block. It should be understood that, in the context of this example, establishing a connection refers to establishing a logical connection over which signals can travel, and does not refer to the creation of an actual physical connection through the installation of wires between the pacing module (104) and the isolator transpolar pen, though in some embodiments such a physical connection might be created, e.g., by closing a switch. Once the surgeon had completed pacing, a signal might be sent from the pacing module (104) to the central processing board (105) indicating that the procedure was complete. The central processing board (105) might then cause the printer module (102) to create hard copy documentation of the procedure which had just been completed. Additionally, the central processing board (105) might use the input/output interface board (106) to send information related to the procedure to some networked storage facility, including local mass storage media for data retrieval.

FIG. 1A is a diagram of an alternate matrix router (10A) which departs from the example of FIG. 1 by utilizing alternate connections between modules (e.g., a direct connection between the central processing board (105) and the printer module (102), instead of only having those modules connected indirectly through the energy generator (101) as was the case in FIG. 1. FIG. 1A also departs from the example of FIG. 1 by incorporating a dedicated sensing module (107) in addition to the pacing module (104) depicted in FIG. 1. It will be appreciated that such a sensing module (107) may, among other things, analyze signals obtained through a device (e.g., an ablation pen) coupled with the matrix router (100) to determine whether fibrillatory signals are crossing over a lesion and/or to provide an indication as to whether the same is occurring. As will be clear to one of ordinary skill in the art, various other combinations and configurations of modules beyond those depicted in FIGS. 1 and 1A could be incorporated into a matrix router (100) without departing from the scope of spirit of the invention.

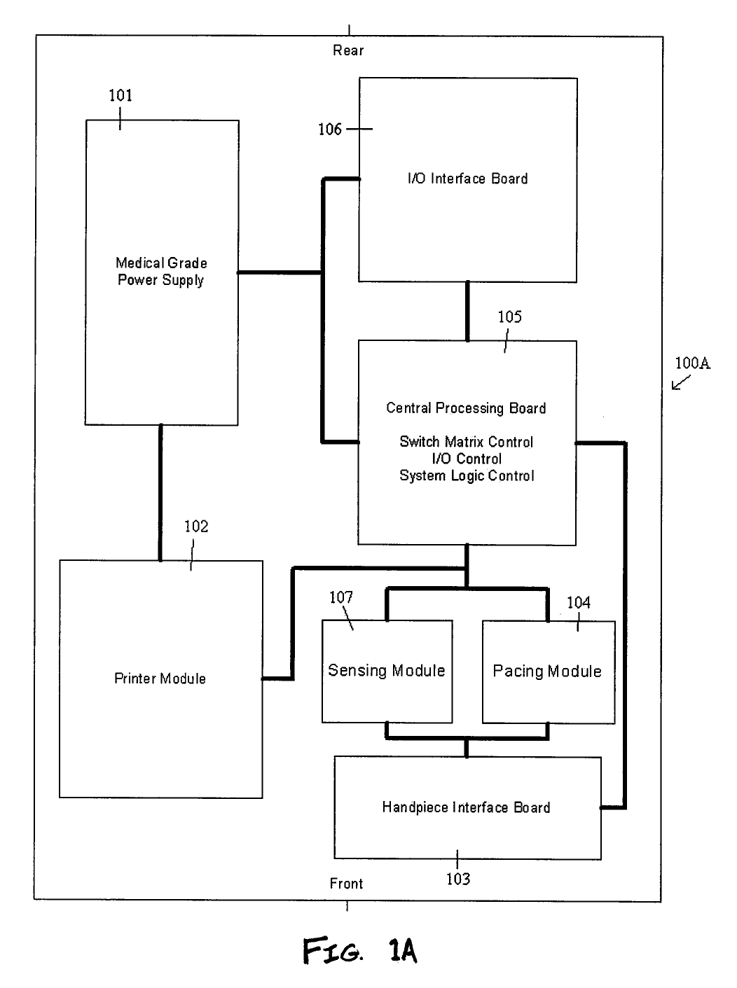

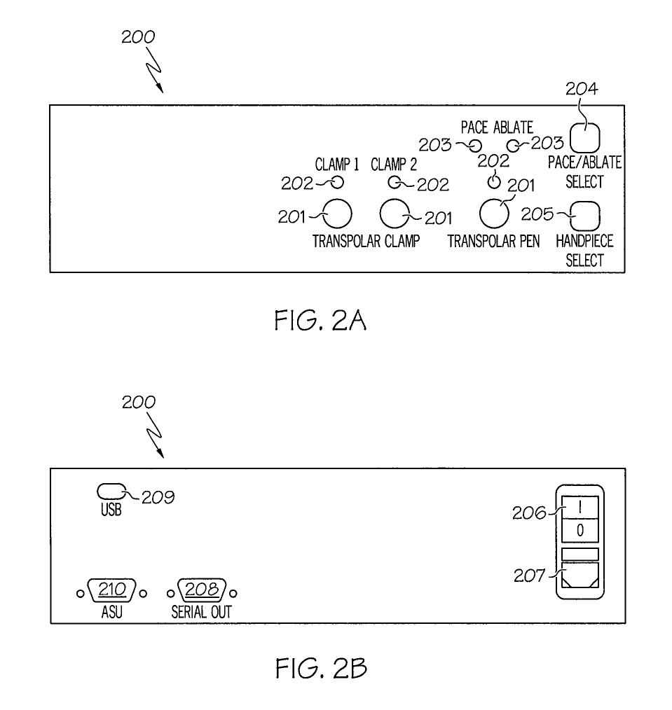

FIGS. 2A and 2B illustrate an exemplary matrix router (200). The front of the matrix router (200) could be used by an operator, even an operator wearing typical surgical garb such as gloves, to switch between different handpieces which might be disposable devices, and different functions, without necessarily having to change handpiece connectors or utilize multiple pieces of capital equipment. The front of the matrix router (200) shown in FIG. 2A comprises multiple interface ports (201) which may be used to establish connections with disposable devices such as an isolator transpolar clamp, an isolator transpolar pen, or any other device. An interface port should be understood to include a location where a connection between one or more devices and/or their constituent components can be established to allow electrical or other signals (e.g., electric current) to pass to or from, or both, the device and/or their components. The matrix router (200) shown in FIG. 2A further comprises activity lights (202) over each interface port (201) which might be used to indicate whether that interface port (201) is currently active and/or for other purposes. In addition to the activity lights (202) over the interface ports (201), the matrix router (200) further comprises mode lights (203), which can be used to indicate whether a device is currently operable in ablate or pace mode, though additional modes (e.g., stimulation mode, sensing mode, etc.) with corresponding mode lights might be utilized in some embodiments. The matrix router (200) further comprises an interface button (205) and a mode button (204) which can be used to change which interface port (201) is active, or which mode a device is to be used in, respectively. An interface port (201) which is active should be understood to mean an interface port (201) which is receiving or transmitting a signal from or to the matrix router (200). For example, if the interface button (205) was used to establish a connection between an energy generator and a first interface port (201), such that energy is being transmitted to a device through the first interface port (201), the first interface port (201) would be said to be active.

In one exemplary use, the matrix router (200) is coupled with an isolator transpolar pen to perform the Maze procedure. Initially, the surgeon might press the interface button (205) until the activity light (202) over the interface port (201) for the isolator transpolar pen is lit. Next, the surgeon might press the mode button (204) until the mode light (203) indicates that the isolator transpolar pen is ready for use in ablation mode. Those lights (202, 203) being lit may signify that there is a connection between an energy generator generating bipolar radio frequency energy and the isolator transpolar pen, and that the pen may therefore be used in ablation mode. Referring to the schematic of FIG. 1., this may be accomplished internally by circuitry comprising the handpiece interface circuit (103), sending a signal to the control circuit (105), requesting a connection be established between the appropriate interface port (201) and an energy generator for generating bipolar radio frequency energy, which might be the energy generator (101), or might be some external generator or other energy source (not shown in FIG. 1). Alternatively, a matrix router (200) could be implemented as a mechanical device wherein the interface of FIG. 2A would establish connections between handpieces and the appropriate energy generators, and the matrix router (200) would remain passive, acting only as a pass-through for signals between the energy generators and handpieces. Further, some embodiments might function using a combination of circuitry and mechanical switches.

While the surgeon is using an isolator transpolar pen to ablate cardiac tissue, the actual amount of bipolar radio frequency energy delivered by the pen might be controlled by operational logic circuitry in the control circuit (105) which might deliver a trigger signal to the energy generator (101) to determine a power generation curve to follow as appropriate for the active device (various power generation curves and methods for selecting them are disclosed in U.S. patent application Ser. No. 11/037,810, filed Jan. 18, 2005 the teaching of which is incorporated by reference herein), or by some external RF generator (not shown). As used herein, an operational logic circuitry should be understood to mean circuitry which specifies one or more outputs on the basis of one or more given inputs. Alternatively, the device being used to ablate tissue, in this case an isolator transpolar pen, might itself generate an identification signal indicating an appropriate power generation curve, and that signal might be translated through the matrix router (200) to the energy generator (101) or some external RF generator, in which case the matrix router (200) might act as a simple pass-through. In some embodiments, an energy generator (101) or an external RF generator might include various operational logic circuitries which would supply power for an appropriate power generation curve, the power generation curve being determined by the identification signal. For example, there might be two defined power generation curves, in which case the energy generator (101) or an external RF generator might contain two operational logic circuitries, one for each power generation curve. Other suitable configurations will be apparent to those of ordinary skill in the art.

Once the surgeon has finished creating a conduction block, he or she might use the pacing module (104), sensing module (107), or other modules which might be incorporated into the matrix router (200) to verify that the tissue making up the block could not transmit electrical signals introduced by pacing the tissue. The matrix router (200) facilitates this switching from ablation to pacing through the use of the mode button (204). Specifically, when the surgeon has finished ablation, he or she could simply press the mode button (204), or request that an assistant press the mode button (204), and the matrix router (200) would switch the isolator transpolar pen from ablation mode to pacing mode. The matrix router (200) as shown in FIG. 2A would provide visual confirmation that the isolator transpolar pen was in the proper mode by extinguishing the mode light (202) indicating ablation, and illuminating the mode light (202) indicating pacing. Referring to the schematic of FIG. 1, mode switching could be accomplished internally by the handpiece interface board (103) establishing a connection between the isolator transpolar pen and the pacing module (104), which would provide electrical signals to stimulate the cardiac tissue, and may further analyze the response detected by the transpolar pen. Additionally, the handpiece interface board (103) or pacing module (104) might also command the control circuit (105) to establish a connection between some external pacing module (not shown in FIG. 1), and the handpiece interface board (103).

While the front side of the matrix router (200) could be used to provide an interface for a surgeon to switch between different devices and different modes, the back of the matrix router (200), as shown in FIG. 2B, might be utilized for other purposes. For instance, the back of the matrix router (200) of this example has an on/off switch (206), together with an input (207) for connecting the matrix router with an external energy source (e.g., a standard wall outlet). The matrix router (200) of this example further comprises a serial input/output port (208) and a USB input/output port (209) (though some embodiments might include multiple serial input/output and/or USB input/output ports) which could be used for data transmission, connecting additional devices, or other purposes. The functionality of those components could be useful for surgery, for example to transmit reports of the procedure, or to create data archives. The matrix router (200) further comprises an interface (210) for an ablation and sensing unit (ASU), which is a piece of capital equipment capable of producing or regulating energy for ablation of tissue and might additionally include operational logic circuitries for following specific output functions for power generation, or provide sensing of various electrical parameters, among other features.

While FIGS. 1, 2A, and 2B depict a schematic of the internal workings and interfaces of an exemplary matrix router (200), those figures are intended to be illustrative only and numerous modifications and variations of the matrix router (200) will be immediately apparent to one of skill in the art. For example, while the example of using a matrix router (200) to facilitate performance of the Maze procedure included a surgeon switching between handpieces using an interface button (205), other embodiments might expand on the handpiece interface circuit (103) of FIG. 1 to enable the matrix router (200) to automatically detect what device is being used by a surgeon, and establish a connection between that device and the appropriate capital equipment (such as the ASU) without needing to be directed by a surgeon using an interface button (205). For instance, the handpiece interface circuit (103) may automatically detect the coupling of a device to any interface port (201) and/or detect the type of device coupled to an interface port (201). Further, it will be appreciated that any other types of data connection may be provided in addition to or in lieu of the serial input/output port (208) and the USB input/output port (209) depicted in FIG. 2B. For example, in addition to, or as an alternative to, the ports (208, 209, 210) depicted in FIG. 2B, a matrix router (200) might have a firewire communications port or a port for a mass storage device such as a flash memory element as well as a wireless communication media. It will be apparent to one of ordinary skill in the art that such ports may be added to the matrix router without departing from the spirit or scope of the invention. Other variations will be apparent to those of ordinary skill in the art.

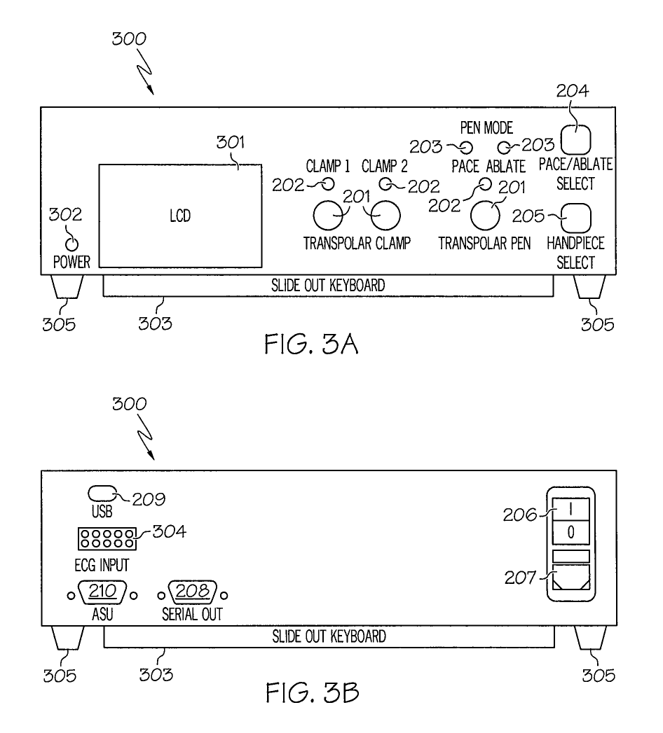

FIGS. 3A and 3B show an alternative matrix router (300). In this example, all the components shown in FIGS. 2A and 2B are present, but additional components, such as a liquid crystal display (LCD) screen (301), a power indicator (302), a keyboard (303), and an input (304) for an ECG and/or esophageal probe or other type of diagnostic or other type of device have been added. In order to illustrate the use of these additional components, consider again the scenario of a surgeon performing the Maze procedure. Using the matrix router (300) the surgeon could follow the procedure outlined above for FIGS. 2A and 2B, but could additionally utilize an ECG, through the input (304) for monitoring the patient's heartbeat to ensure that the procedure was successful. Additionally, the surgeon could use the LCD screen (301) to monitor the ECG, avoiding the necessity of having a separate piece of display equipment. The slide out keyboard (303) would allow the surgeon (or an assistant, as appropriate), to input data such as patient demographics and/or physical characteristics into the matrix router (300). These additional data sources, the keyboard (303) and the input (304) may allow a more complete picture of the operation to be created, which could be archived using the serial input/output port (208) or the USB input/output port (209). The entered information can also be printed and hardcopy made available for patient record. Additionally, the keyboard (303) might be used for system configuration or other purposes, while the LCD screen (301) could be used for data presentation, in addition to simply displaying the ECG output. The power indicator (302) of this example comprises a light that is illuminated when the matrix router (300) is drawing power from an energy source (not shown). Matrix router (300) further comprises legs (305) which would allow the matrix router (300) to be placed on top of another piece of equipment, such as an ASU, without interfering with the use of the slide out keyboard (303).

As with FIGS. 1, 1A, 2A, and 2B, FIGS. 3A and 3B are intended to be illustrative only of certain components which could be added to a matrix router (300) in addition to those shown in FIGS. 2A and 2B. Various modifications and alterations to the components shown in FIGS. 3A and 3B will be immediately apparent to one of ordinary skill in the art. For example, the LCD screen (301) of FIG. 3A could be replaced with an alternative display technology, such as a cathode ray tube (CRT) monitor, or a plasma screen monitor, or could even be moved out of the matrix router all together, and replaced with a connection to an external graphic display device. Similarly, it will be immediately apparent to one of ordinary skill in the art that, instead of having an input to an external ECG or other diagnostic device (304), an internal ECG or other diagnostic device could be integrated into the matrix router itself. Thus, it should be understood that FIGS. 3A and 3B, like the figures which preceded them, are intended to be illustrative only, and not limiting.

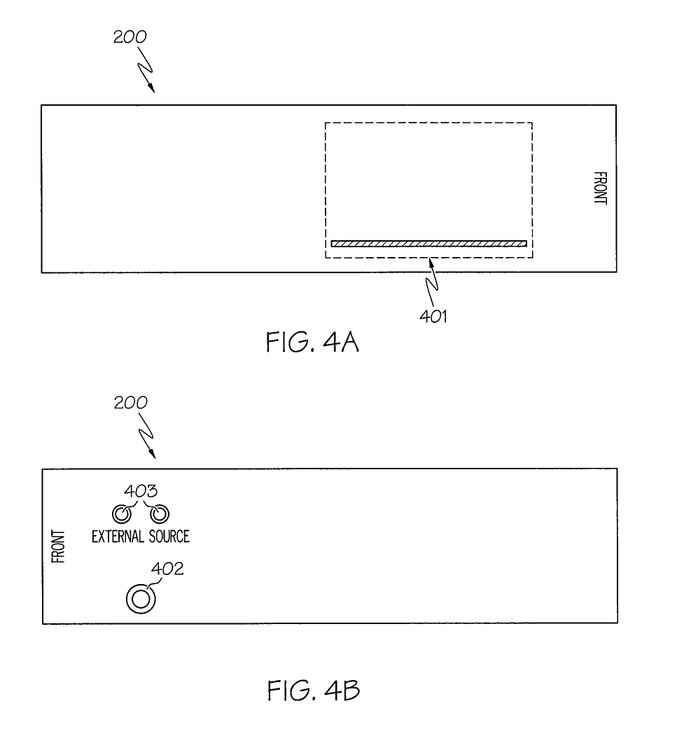

FIGS. 4A and 4B illustrate side views of the exemplary matrix router (200) of FIGS. 2A and 2B, with FIG. 4A illustrating a left view, and FIG. 4B illustrating a right view. In this embodiment, the sides of the matrix router (200) are used for input and output ports. For example, the left view of FIG. 4A includes a printer module (401), which could be a thermal printer or other type of printer integrated with the matrix router (200). The printer module (401) could be used to provide hard copy confirmation and documentation of procedures which were performed utilizing the matrix router (200). Other information suitable for printing by printer module (401) will be apparent to those of ordinary skill in the art. In the right view of FIG. 4B, there is both an interface (402) for a cable connected to an ASU or other device, and ports (403) for a connection to an alternative external energy or data source. Matrix router (200) may further comprise additional buttons or other features for switching between energy or data sources, in the same manner as the interface button (205) shown in of FIGS. 2A and 3A allows switching between multiple handpieces. Indeed, in some embodiments, there might be only a single interface port (201), which may allow multiple pieces of equipment, such as different generators, to drive a single disposable device, such as an isolator transpolar pen. In one embodiment, a foot switch (not shown) is coupled with the matrix router (200) to initiate delivery of RF energy as an example. Such a foot switch could be used to substitute or supplement the interface button (205), the mode button (204), and/or provide any other suitable features. In yet another embodiment, a substitute or supplement for the interface button (205) and/or the mode button (204) is provided in a surgical device (not shown) coupled with the matrix router (200). In this embodiment, the matrix router (200) is operable to detect selections made by such a feature on the surgical device, and is configured to provide a signal to the surgical device in accordance with such selections.

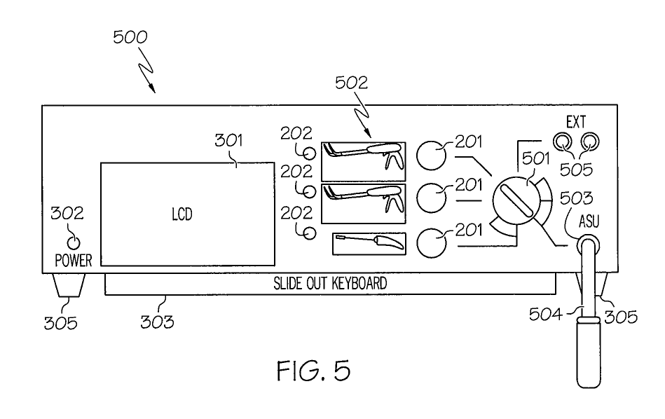

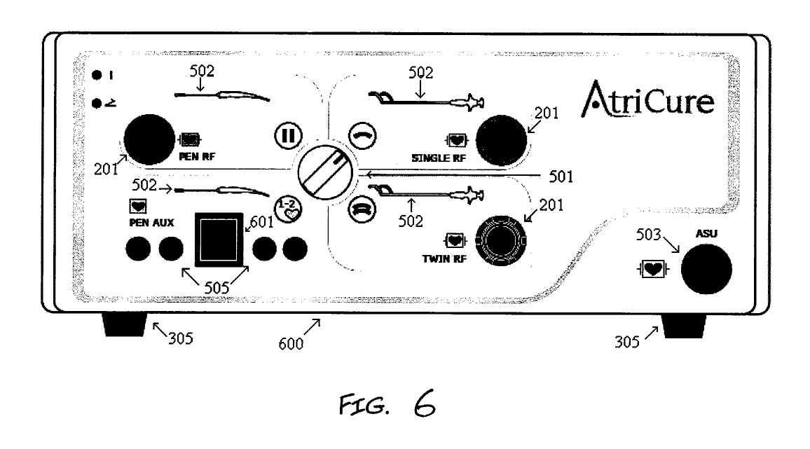

FIG. 5 shows a front view of another alternative matrix router (500). In this example, the interface and mode buttons (204, 205) shown in FIGS. 2A and 3A have been replaced with a single selection dial (501) which is operable to control which interface port (201) is to be connected to an ASU (not shown) or to another device. Additionally, the matrix router (500) of this example includes pictorial indications (502) of devices for each interface port (201), increasing the convenience of using the matrix router (500). The matrix router (500) further comprises an interface (503) for connecting a cable (504) to an ASU, as well as additional ports (505) for connecting an additional external power source or other external device. In this way, matrix router (500) combines the interface functionality illustrated in FIGS. 2A and 3A, with the power interface components shown in FIG. 4B.

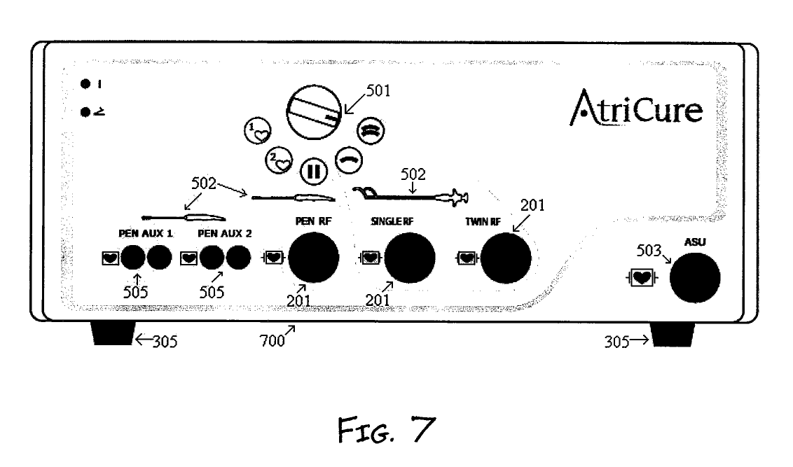

FIG. 6 shows a front view of another alternative matrix router (600) having many of the features described in relation to previous diagrams, such as a selection dial (501), interface ports (201), an interface (503) for connecting to an ASU, as well as other ports (505) for connecting to additional external power sources or other external devices. However, while there are similarities between the matrix router (600) depicted in FIG. 6 and those depicted previously, there are also some differences. One such difference is that, the matrix router (600) depicted in FIG. 6 includes two additional ports (505) for connecting to additional external power sources or other external devices. Those additional ports (505) could be used to simplify the performance of procedures which utilize additional pieces of external equipment. For example, in the matrix router (600) of FIG. 6, a dedicated ablation unit could be connected to the interface (503), and separate pacing and sensing units could be connected to the additional ports (505). When utilizing the pacing or sensing units, the surgeon could switch to the appropriate additional port (505) using the switch (601) between those ports (505). This might simplify workflow by allowing a surgeon to alternate between multiple pieces of additional equipment (the pacing and sensing units) by using a switch (601) rather than by disconnecting one piece of equipment so that the other could be connected to the single additional port (505). Of course, as will be apparent to one of ordinary skill in the art, the invention is not restricted to the numbers or configurations of ports depicted in the diagrams, and some embodiments of the invention will include matrix routers with more ports, or ports in alternate configurations, or both. For example, FIG. 7 depicts a matrix router (700) in which switching between additional ports (505) is performed using a selection dial (501) rather than with a dedicated switch as in FIG. 6.

While FIGS. 5-7 demonstrate one particular alternate means of switching handpiece connections, the selection dial (501), it will be apparent to one of ordinary skill in the art that there are many additional features, such as levers, sliders, switches, etc., which could be used to select handpiece connections. Further, it will be immediately apparent to one of ordinary skill in the art that various other components, such as a modem which could be used for remote system diagnostics or data transmission, or a fax which could be used for local transmission of full disclosure, could easily be added to a matrix router (200, 300, 500, 600, 700) and that such augmented matrix routers are well within the scope of the invention. Further, one of ordinary skill in the art will immediately recognize that virtually any component of a matrix router (200, 300, 500, 600, 700) could be integrated into the matrix router (200, 300, 500, 600, 700) itself, or could be attached to the matrix router (200, 300, 500, 600, 700) via an interface port. To illustrate this option, the following table sets forth component configurations for a number of embodiments, and also indicates that different embodiments might have different combinations of integrated and externally provided components.

TABLE 1

| | | Handpiece | Handpiece Switching | Handpiece Switching | Handpiece Switching | Handpiece Switching with |
|---|---|---|---|---|---|---|
| | Handpiece | Switching with | with External Pace, | with External Pace, | with Integrated | Integrated Pace, Sense, |
| | Switching | External Pace | Sense, and Stimulate | Sense, and Stimulate | Pace, Sense | Stimulate Circuits plus |
| | Only | Input Source | Input Sources | Input Sources | Stimulate Circuits | [symbol] |
| Transpolar Clamp | E | E | E | E | E | E |
| Transpolar Pen | E | E | E | E | E | E |
| Pace Module | | E | E | E | X | X |
| Sense Module | | | E | E | X | X |
| Stimulate Module | | | E | E | X | X |
| Patient ECG | | E | E | E | X | X |
| Thermal Printer | | X | X | X | X | X |
| Graphical Display/ | | | | X | | X |
| KB | | | | | | |
| Modem | | | | X | | X |
| Mass Storage | | | | X | | X |

E = Externally provided to the matrix router
X = Integrated with the matrix router

Of course, the configurations shown in Table 1 are merely exemplary. Still other ways in which features may be allocated integrally and externally will be apparent to those of ordinary skill in the art.

In addition to simplifying the use of various surgical devices as set forth above, certain embodiments of the matrix router (200, 300, 500, 600, 700) might additionally be configured to automatically document the use of the matrix router (200, 300, 500, 600, 700). For example, in some embodiments, the matrix router (200, 300, 500, 600, 700) might automatically compile a record of which interfaces and/or which modes were activated throughout the course of a surgical procedure. Similarly, in some embodiments which include data inputs, such as an ECG, the matrix router (200, 300, 500, 600, 700) might automatically compile information provided by those data inputs as well. Such data compilation might be further integrated with data provided through the keyboard, or might be used as an additional or alternative source of documentation for a surgical procedure.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometries, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A surgical router, comprising:
a) an energy source operable to power a plurality of surgical devices;
b) a plurality of interface ports operable to connect the plurality of surgical devices to the router, the plurality of surgical devices comprising:
(i) a first ablation surgical device,
(ii) a second surgical device; and
c) a switch operable to selectively connect the energy source to the first ablation surgical device or the second surgical device.

2. The surgical router of claim 1, wherein the first ablation surgical device comprises one or more of:
i) an ablation clamp, or
ii) an ablation pen.

3. The surgical router of claim 1, further comprising:
(a) a plurality of operational logic circuitries operable to regulate energy from the energy source;
(b) a selection circuit operable to selectively activate an operational logic circuitry of the plurality of operational logic circuitries.

4. The surgical router of claim 1, wherein the energy source is operable to provide radio frequency energy.

5. The surgical router of claim 4, wherein the radio frequency energy is bi-polar.

6. The surgical router of claim 4, wherein the radio frequency energy is mono-polar.

7. The surgical router of claim 1, wherein the energy source is operable to provide energy from the group consisting of ultrasonic energy, microwave energy, and laser energy.

8. The surgical router of claim 1, wherein the switch is manually operable.

9. A surgical router, comprising:
a) an interface port operable to connect with a first surgical device, the first surgical device being operable in a set of modes, the set of modes comprising:
i) a pacing mode,
ii) a sensing mode, and
iii) an ablation mode;
b) an energy source operable to transmit a power signal via the interface port to the first surgical device in the ablation mode;
c) a pacing module in communication with the energy source, wherein the pacing module is operable to transmit a pacing signal to the first surgical device in the pacing mode; and
d) a first switch operable to selectively enable transmission of:
i) the power signal, or
ii) the pacing signal
to the first surgical device.

10. The surgical router of claim 9, further comprising
(a) a second interface port operable to connect with a second surgical device; and
(b) a second switch operable to selectively enable transmission of the power signal to the first surgical device or the second surgical device.

11. The surgical router of claim 10, wherein the first switch is the same as the second switch.

12. The surgical router of claim 9, wherein the first surgical device comprises an ablation pen.

13. The surgical router of claim 9, further comprising:
(a) a plurality of operational logic circuitries operable to regulate energy from the energy source; and
(b) a selection circuit operable to selectively activate an operational logic circuitry of the plurality of operational logic circuitries.

14. The surgical router of claim 9, wherein the energy source is operable to provide radio frequency energy.

15. A surgical router, comprising:
a) an energy source;
b) a first interface port operable to connect with a first surgical device;
c) a plurality of operational logic circuitries in communication with the energy source and the first interface port comprising:
(i) a first operational logic circuitry operable to implement a first power generation curve by regulating energy from the energy source, and
(ii) a second operational logic circuitry operable to implement a second power generation curve, by regulating energy from the energy source; and
d) a circuitry operable to selectively activate an operational logic circuitry from the plurality of operational logic circuitries.

16. The surgical router of claim 15 further comprising:
a) a second interface port operable to connect with a second surgical; and
b) a switch operable to selectively connect the energy source to the first surgical device or the second surgical device.

17. The surgical router of claim 15 further comprising a first switch operable to selectively connect the first surgical device to:
(i) the energy source, or
(ii) a pacing module, wherein the pacing module is in communication with the energy source, wherein the pacing module is operable to provide a pacing signal.

18. The surgical router of claim 17, further comprising:
a) a second interface port for connecting with a second surgical device;
b) a second switch operable to selectively connect the energy source to the first surgical device or the second surgical device.

19. The surgical router of claim 15, wherein the first surgical device comprises one of:
(a) an ablation pen; or
(b) an ablation clamp.

20. The surgical router of claim 15, further comprising a data compilation module operable to automatically compile data related to a surgical procedure.
